(19) United States
(12) Patent Application Publication
(10) Pub. No.: US 2025/0018184 A1
(43) Pub. Date: Jan. 16, 2025

(54) DEVICE FOR TREATING SLEEP APNEA USING HIGH-INTENSITY FOCUSED ELECTROMAGNETIC AND RADIO FREQUENCY TECHNOLOGIES

(71) Applicant: Agileworks LLC, Alexandria, VA (US)

(72) Inventor: Jonathan HAASE, Alexandria, VA (US)

(73) Assignee: Agileworks LLC, Alexandria, VA (US)

(21) Appl. No.: 18/774,273

(22) Filed: Jul. 16, 2024

Related U.S. Application Data
(60) Provisional application No. 63/527,051, filed on Jul. 16, 2023.

(51) Int. Cl. A61N 1/36

(52) U.S. Cl. CPC A61N 1/3601

(57) ABSTRACT

Embodiments provide device for treating sleep apnea, the device including: a stand, a chin and neck support, an applicator integrated into the chin and neck support configured to provide High-Intensity Focused Electromagnetic Technology (HIFEM) therapy and Radio Frequency (RF) therapy, and a control panel configured to adjust an intensity of the delivered HIFEM and RF therapies in real-time. The applicator is configured to provide HIFEM therapy by delivering HIFEM energy to a patient's mouth, throat, and neck to stimulate and strengthen muscles associated with the patient's breathing, and further configured to provide RF therapy by delivering RF energy to reduce fat tissue in the patient's airway.

### CROSS REFERENCE TO RELATED APPLICATION

This application claims priority to U.S. Provisional Patent Application Ser. No. 63/527,051, filed on Jul. 16, 2023, which is hereby incorporated by reference in its entirety into this application.

### BACKGROUND

### Field

Embodiments generally relate to a device for treating sleep disorders. More particularly, embodiments of the disclosure relate to a medical device for treating sleep apnea using High-Intensity Focused Electromagnetic Technology (HIFEM) and Radio Frequency (RF) Technology.

### Description of the Related Art

Sleep apnea is a common disorder characterized by repeated interruptions in breathing during sleep, leading to poor sleep quality and various health risks. Current treatments for sleep apnea include lifestyle modifications, use of oral appliances, Continuous Positive Airway Pressure (CPAP) devices, and surgical interventions. However, these treatments often present challenges, such as discomfort, inconvenience, non-compliance, or side effects.

Despite the availability of various treatments, many patients with sleep apnea continue to experience symptoms. Sleep apnea could be caused by non-compliance with treatment, ineffective current solutions which are uncomfortable and disrupt sleep due to hose entanglements and air leaks, making sleeping painful and difficult for patients, or intolerance of side effects.

Various approaches have been developed to address widespread sleep disorders, such as obstructive sleep apnea (OSA). Many of these approaches can be broadly categorized into several groups: (1) methods and devices that maintain patency of the upper airway through positive pressure or physical support, (2) systems that monitor and control sleep positions or other physical parameters, (3) implants, or (4) surgical interventions, among others.

For example, JP5738879B2 by Russo Robert Ei and Ethicon Inc., U.S. Pat. No. 8,037,885B2 by Anja K. Metzger and Restore Medical Inc., and JP5859446B2 by Kevin S. Weedock and Ethicon Inc. describe solutions focused on implants or mechanical alterations to the airway. These medical approaches leverage implanted devices in the soft tissue or submandibular region to maintain airway patency, a strategy requiring invasive procedures and carrying associated surgical risks.

U.S. Pat. No. 7,322,356B2 by Susan L. Critzer and Restore Medical Inc., CN107635615B by R. Evers & Fresca Medical Company, and U.S. Pat. No. 6,488,634B1 by David M. Rapoport & New York University focus on pressure adjustments and airflow control, usually via external devices that provide positive airway pressure. Although these medical approaches are non-invasive, patients often struggle with compliance due to discomfort or inconvenience.

U.S. Pat. No. 10,500,085B2 by Men-Tzung Lo and National Central University and CN109310348B by T. G. Goff and Hancock Medical Company, propose monitoring and detection systems for OSA. These systems are usually non-invasive but primarily serve for diagnosis and monitoring rather than direct treatment of OSA.

Other methods and devices for HIFEM aesthetic treatment of biological structures have been developed. HIFEM has been used to increase muscle size and reduce fat, but none of these prior methods and devices relate to the mouth, throat, and/or neck and none have been shown to treat sleep apnea.

Other methods and devices have been developed for aesthetic treatments, including using magnetic fields and radio frequency energy to treat biological structures, unattended patient treatment methods and devices for inducing lipolysis. However, none of these treatments address sleep apnea.

What is needed is a more effective, comfortable, and convenient treatment for sleep apnea, ideally one that is administered clinically with little or no effort on the part of the patient.

### SUMMARY

Embodiments provide an industrial-grade device that uses HIFEM and RF technologies to treat sleep apnea. The device strengthens the muscles involved in breathing, for example, in the mouth, tongue, neck and throat, and reduces fat in the airway, alleviating sleep apnea symptoms. The device offers a non-invasive, effective, and comfortable treatment for sleep apnea. It can be used in a clinical setting, and the treatment can be customized to the needs of each patient. The device according to various embodiments may be used alone to treat sleep apnea, or it can be combined with other traditional sleep disorder treatment devices or methods, such as a CPAP device or therapy to improve patient outcomes.

Embodiments provide a device and a method for strengthening the muscles of respiration in a clinical, controlled approach allowing for gradual increases of intensity over time to achieve a reduction or reversal of OSA, similar to the way manual exercises can reduce sleep apnea. The device and method, according to various embodiments, use HIFEM energy to strengthen the muscles that control breathing, for example, by inducing supramaximal muscle contractions, which can improve muscle density and volume, in a safe, comfortable, clinical environment with the use of RF to generate heat for reducing fat cells contributing to OSA.

The device and the method according to various embodiments are novel and distinct from the conventional medical approaches because they address the underlying muscular weakness often observed in patients with OSA. The device and the method strengthen the muscles associated with maintaining airway patency, i.e., stimulating and strengthening the muscles involved in breathing and reducing fat in the patient's airway, thereby increasing the size of the airway and preventing the tongue from obstructing breathing during sleep. This offers a unique, non-invasive strategy that addresses the root cause of sleep disorders, such as sleep apnea, rather than just managing symptoms.

Thus, the device and the method according to various embodiments minimizes discomfort and disruption and enhances compliance compared to positive airway pressure devices. Furthermore, the device and the method avoid invasive surgical procedures required by many implant-based solutions. The device and the method improve the patient's muscle tone and reduces fat constricting the patient's airway, enhancing natural physiological processes and providing a more holistic approach to managing OSA.

Embodiments provided a device for treating sleep apnea. The device includes a stand, a chin and neck support, an applicator integrated into the chin and neck support configured to provide High-Intensity Focused Electromagnetic Technology (HIFEM) therapy and Radio Frequency (RF) therapy, and a control panel configured to adjust an intensity of the delivered HIFEM and RF therapies in real-time. The applicator is configured to provide HIFEM therapy by delivering HIFEM energy to a patient's mouth, throat, and neck to stimulate and strengthen muscles associated with the patient's breathing, and is further configured to provide RF therapy by delivering RF energy to reduce fat tissue in the patient's airway.

According to an embodiment, the chin and neck support includes a stem portion and a seat portion, the stem portion of the chin and neck support configured to support the patient's neck and the seat portion of the chin and neck support configured to support the patient's jaw during treatment.

According to an embodiment, the device further includes one or more HIFEM and RF generators, each HIFEM generator configured to generate HIFEM energy for providing the HIFEM therapy to the patient's mouth, throat and neck, and each RF generator configured to generate RF energy for providing RF therapy to the patient's airway.

According to an embodiment, the applicator is configured to deliver the HIFEM and RF energy concurrently to the muscles and fat tissues in the airway of the user of the device.

According to an embodiment, the chin and neck support is selected based on the length and width of the user's neck and jaw to enhance the user's comfort and the effectiveness of treatment.

According to an embodiment, the applicator is configured to concurrently deliver the HIFEM energy pulsed at between 1 Hz to 500 Hz with a continuous application of RF energy in the range of 1 MHz to 3 MHZ.

According to an embodiment, the chin and neck support and the applicator are each curved to maintain a uniform distance to the neck throughout an HIFEM and RF energy application zone.

According to an embodiment, the control panel includes a patient-operated button configured to terminate the delivery of the HIFEM and RF energy, when the patient experiences discomfort during treatment.

According to an embodiment, the applicator is configured to deliver the HIFEM energy and RF energy for a duration of 10 to 30 minutes long, depending on the patient's comfort level.

According to an embodiment, there is also provided a method for treating sleep apnea, the method using a device. The device includes a stand, a chin and neck support, an applicator integrated into the chin and neck support configured to provide High-Intensity Focused Electromagnetic Technology (HIFEM) therapy and Radio Frequency (RF) therapy, and a control panel configured to adjust an intensity of the delivered HIFEM and RF therapies in real-time, wherein the applicator is configured to provide HIFEM therapy by delivering HIFEM energy to a patient's mouth, throat, and neck to stimulate and strengthen muscles associated with the patient's breathing, and further configured to provide RF therapy by delivering RF energy to reduce fat tissue in the patient's airway. The method includes the steps of selecting the chin and neck support based on the length and width of the user's neck and jaw to enhance the user's comfort and the effectiveness of treatment, generating the HIFEM and RF energy using a set of HIFEM and RF generators, delivering the HIFEM energy from the applicator to the patient's mouth, throat, and neck through the patient's jaw to stimulate and strengthen muscles associated with the patient's breathing, concurrently delivering the RF energy from the applicator to the patient's neck to reduce fat tissue in the patient's airway, and adjusting the intensity of the delivering of the HIFEM energy and the RF energy based on the patient's comfort, using the control panel to treat the patient's sleep apnea.

According to an embodiment, the delivering of the HIFEM energy is pulsed at between 1 Hz to 500 Hz with the delivering of a continuous application of RF energy in the range of 1 MHz to 3 MHz for 30 minutes.

According to an embodiment, the method further includes the step of terminating the delivery of the HIFEM energy and the RF energy, using a patient-operated button on the control panel, when the patient experiences discomfort during treatment.

According to an embodiment, the method further includes the step of treating the patient's sleep apnea using a Continuous Positive Airway Pressure device.

### BRIEF DESCRIPTION OF THE DRAWINGS

So that the manner in which the recited features, aspects and advantages of the disclosure, as well as others that will become apparent, are attained and can be understood in detail, a more particular description of certain embodiments briefly summarized above can be had by reference to the embodiments that are illustrated in the drawings that form a part of this specification. It is to be noted, however, that the appended drawings illustrate only exemplary embodiments and are, therefore, not to be considered limiting of the disclosure's scope, for the disclosure can admit to other equally effective embodiments. Like numbers refer to like elements throughout, and the prime notation, if used, indicates similar elements in alternative embodiments or positions.

FIG. 1 is a side view of a device for treating sleep apnea, according to an embodiment.

FIG. 2 is a side view of a chin and neck support of a device for treating sleep apnea, according to an embodiment.

FIG. 3 is a side view of a stand, a base, and a chin and neck support of a device for treating sleep apnea, according to an embodiment.

FIG. 4 is a top view of a chin and neck support and an applicator configured to deliver HIFEM and RF energy, according to an embodiment.

FIGS. 5A-5C illustrate a front, side, and rear view of a chin and neck support and an applicator of a device for treating sleep apnea, according to an embodiment.

FIG. 6 illustrates a method for treating sleep apnea, according to an embodiment.

### DETAILED DESCRIPTION

While the scope of the system and method will be described with several embodiments, it is understood that one of ordinary skill in the relevant art will appreciate that many examples, variations and alterations to the systems and methods described here are within the scope and spirit of the embodiments.

Accordingly, the embodiments described are set forth without any loss of generality, and without imposing limitations, on the embodiments. Those of skill in the art understand that the scope includes all possible combinations and uses of particular features described in the specification.

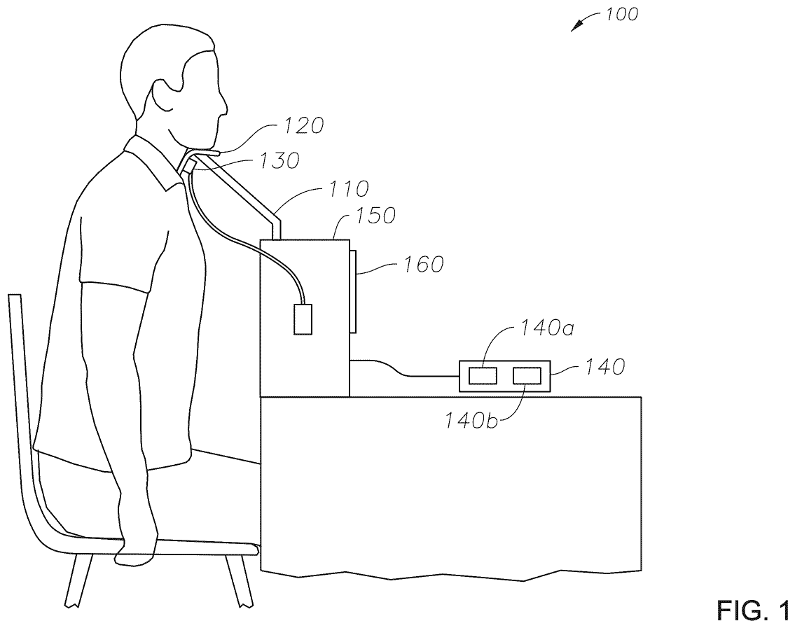

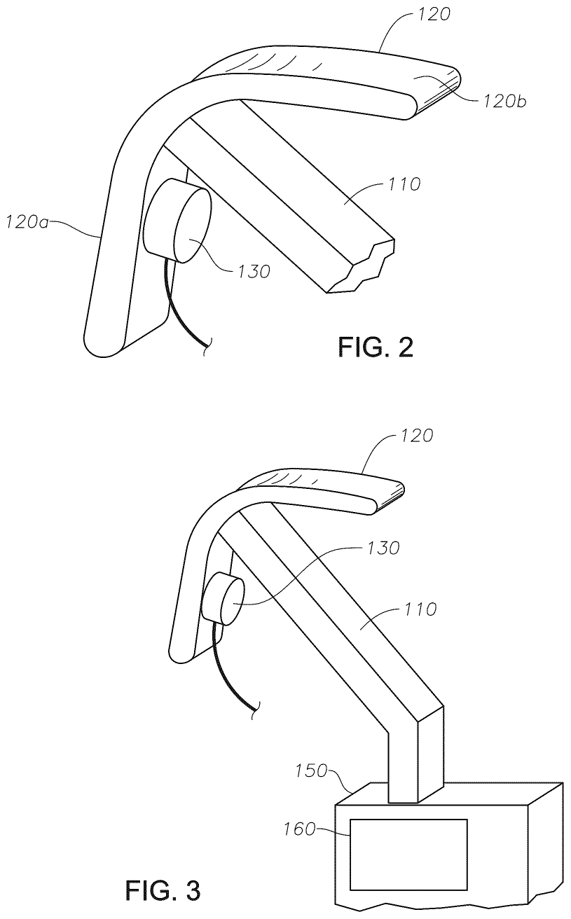

Referring now to the disclosed devices in more detail, FIG. 1 is a side view of a device for treating sleep apnea, according to an embodiment. FIG. 2 is a side view of a chin and neck support of a device for treating sleep apnea, according to an embodiment. FIG. 3 is a side view of a stand, a base, and a chin and neck support of a device for treating sleep apnea, according to an embodiment.

As shown in FIGS. 1 and 3, the device 100 includes a stand 110 including a chin and neck support 120, an applicator 130 integrated into the chin and neck support configured to deliver High-Intensity Focused Electromagnetic Technology (HIFEM) and Radio Frequency (RF) energy (or therapy), and a control system 140 arranged in the stand 110 and configured to adjust an intensity of the delivered HIFEM and RF energy in real-time.

According to an embodiment, the stand 110 serves as the main structure of the device, providing stability and support for the other components. The stand 110 is made from durable materials, that can withstand repeated use in a clinical setting. The stand 110 also includes features for adjusting the height and angle of the device to accommodate patients of different sizes and positions.

According to an embodiment, the chin and neck support 120 is a non-adjustable, one-piece design that serves multiple functions. For example, the chin and neck support 120 is designed to comfortably accommodate the patient's chin and neck, allowing them to comfortably rest on the hard plastic stand 110 during treatment. The applicator 130 is connected to a set of HIFEM and RF generators 140 configured to generate HIFEM and RF energy, respectively, to be concurrently delivered through the applicator 130 to the patient during treatment. The chin and neck support 120 is configured to ensure that the HIFEM and RF energy is directed to the appropriate spots in the patient's mouth and throat to strengthen the muscles of respiration and clear the patient's airway. In operation, the physician will select the appropriate size of the chin and neck support 120 based on the patient's anatomy. Because the chin and neck support 120 is a separate element and comes in varying sizes, the physician can interchange chin and neck supports 120 to accommodate the patient's varying size and to connect with other HIFEM and RF generators 140 configured in the stand 110.

According to an embodiment, the chin and neck support 120 includes the applicator 130 configured to deliver HIFEM and RF energy into the mouth and throat, generated by the set of HIFEM and RF generators 140 supported by the stand 110, as shown in FIG. 1.

According to an embodiment, as shown in FIG. 2, the chin and neck support are configured in a manner which allows a patient to comfortably rest the patient's neck, and specifically the throat, against a stem portion 120a of the chin and neck support 120, while the patient comfortably rests the patient's chin or jaw on a seat portion 120b of the chin and neck support 120.

As further shown on FIG. 1., the stand 110 includes a base 150 configured to house a control panel 160 and provide stability to the stand 110 of the device 100. The control panel 160 allows the operator to control the device 100, adjust the settings for generation and application of the HIFEM and RF energy, and to monitor the progress of the treatment. The control panel 160 includes a set of buttons, a set of dials, and a user touchscreen configured to adjust the intensity mof the application of the HIFEM and RF energy, and/or a display for monitoring the treatment progress.

According to an embodiment, the HIFEM generator 140a is a high-powered component that produces a high intensity focused electromagnetic field used in HIFEM therapy. The HIFEM generator 140a includes features for adjusting the intensity of the energy field, allowing the therapy to be customized for each patient's treatment.

According to an embodiment, the RF generator 140b is a powerful component that produces the radio frequency used in RF therapy. The RF generator 140b includes features for adjusting the intensity of the radio frequency, allowing the therapy to be customized for each patient's treatment.

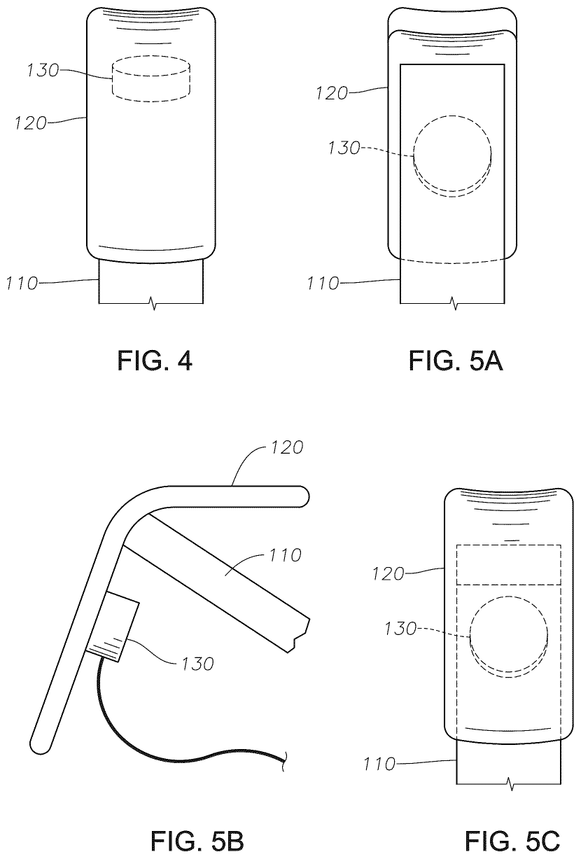

FIG. 4 is a top view of a chin and neck support and an applicator configured to deliver HIFEM and RF energy, according to an embodiment. According to an embodiment, the chin and neck support 120 is made of a hard polymer surface shown by the solid line in FIG. 4. The applicator 130 is placed under the patient's jaw. The set of HIFEM and RF generators 140a, 140b generate HIFEM and RF energy and transmit the HIFEM and RF energy through the applicator 130, shown as the dashed line in FIG. 4, up into the patient's mouth and throat through the bottom of the patient's jaw. The HIFEM and RF energy can penetrate the patient's skin and soft tissues of the mouth and throat through the jaw. The applicator 130 comes in various sizes to accommodate different jaw widths, providing comfort to the patient during treatment.

FIGS. 5A-5C illustrate a front, side, and rear view of a chin and neck support and an applicator of a device for treating sleep apnea, according to an embodiment. According to an embodiment, the chin and neck support and the applicator are each curved to maintain a uniform distance to the neck throughout the HIFEM and RF energy application zone. The HIFEM and RF energy is applied to the patient in the zone, as depicted by the dashed line in FIG. 5A. The curvature configuration of the chin and neck support and the applicator allows each patient to be fitted with a properly sized chin and neck support, ensuring the safe and effective application of HIFEM and RF energy to the neck to strengthen the muscles of the mouth, throat, neck, and airway to reverse sleep apnea by decreasing the fat in the airway, thereby creating a larger breathing passage and increasing the strength of the muscles preventing breathing obstructions during sleep.

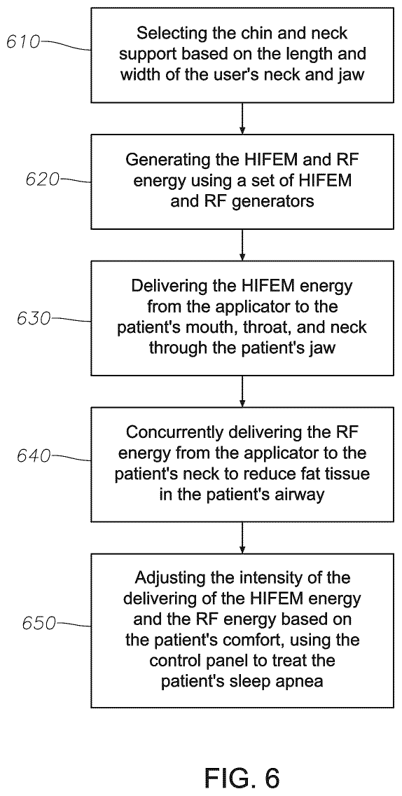

FIG. 6 illustrates a method for treating sleep apnea, according to an embodiment. The method includes the steps of selecting the chin and neck support based on the length and width of the user's neck and jaw to enhance the user's comfort and the effectiveness of treatment (step 610), generating the HIFEM and RF energy using a set of HIFEM and RF generators (step 620), delivering the HIFEM energy from the applicator to the patient's mouth, throat, and neck through the patient's jaw to stimulate and strengthen muscles associated with the patient's breathing (step 630), concurrently delivering the RF energy from the applicator to the patient's neck to reduce fat tissue in the patient's airway (step 640), and adjusting the intensity of the delivering of the HIFEM energy and the RF energy using the control panel to treat the patient's sleep apnea (step 650). The method is performed using a device according to various embodiments, in which the device includes a stand, a chin and neck support, an applicator integrated into the chin and neck support configured to provide HIFEM therapy and RF therapy, and a control panel configured to adjust an intensity of the delivered HIFEM and RF therapies in real-time, wherein the applicator is configured to provide HIFEM therapy by delivering HIFEM energy to a patient's mouth, throat, and neck to stimulate and strengthen muscles associated with the patient's breathing, and further configured to provide RF therapy by delivering RF energy to reduce fat tissue in the patient's airway.

The method for treating sleep apnea according to an embodiment will now be described in more detail.

Initiation of Treatment: The method for treating sleep apnea, according to an embodiment, begins with the patient being fitted for the correct chin and neck support 120 including the HIFEM/RF applicator 130. The HIFEM/RF applicator 130 is designed to extend beyond the width and length of the patient's jaw, providing a comfortable resting surface. According to an embodiment, the HIFEM/RF applicator 130 is a single color with a contrasting outline indicating the area where the HIFEM and RF energy are projected from the HIFEM/RF applicator 130. This design ensures that the patient can comfortably rest their entire jaw on the HIFEM/RF applicator 130, while the HIFEM and RF energy are directed directly into the mouth of the patient through the patient's jaw, thereby targeting the tongue and palate. As previously noted, the HIFEM and RF energy can penetrate the patient's skin and soft tissues of the mouth and throat through the jaw. A similar approach is used for the patient's neck with the stand 110 extending beyond the width of the neck and being rounded to maintain close contact with all frontal points of the patient's neck. The stand 110 also features a contrasting color outline showing where the HIFEM and RF energy will be directed into the patient's throat and neck. In alternate embodiments, the stem portion of the chin and neck support for resting the patient's neck and the seat portion of the chin and neck support for resting the patient's jaw are separate components.

According to an embodiment, a physician or medical practitioner is needed to properly fit the patient to the chin and neck support 120, to operate the device 100, and to monitor the application of the HIFEM and RF energy, to ensure proper delivery of the HIFEM and RF energy to the patient for treatment.

Delivery of HIFEM Therapy: The method further includes the generation of HIFEM energy and delivery of the HIFEM energy via the HIFEM/RF applicator 130 arranged in the chin and neck support 120 to the patient's muscles responsible for controlling breathing. HIFEM therapy is crucial to the process for treating sleep apnea, according to an embodiment. The HIFEM generator 140a produces a high intensity focused electromagnetic field. The electromagnetic field is carefully directed toward the muscles involved in breathing, specifically the mouth, throat, and neck. The HIFEM therapy induces supramaximal muscle contractions, which are impossible through voluntary contractions. When exposed to these strong contractions, the muscle tissue adapts to extreme conditions by remodeling its inner structure, resulting in muscle building and sculpting. In the context of sleep apnea treatment, the HIFEM therapy targets and strengthens the muscles involved in breathing. These muscles include, for example, the genioglossus muscle, which controls the tongue's position, and the pharyngeal muscles, which form the walls of the throat. By strengthening these muscles, the device improves the muscles' ability to keep the airway open during sleep, reducing the likelihood of apnea events. The intensity of the HIFEM field can be adjusted using the control panel, allowing the therapy to be customized for each patient.

In practice, the physician or medical practitioner increases or decreases the intensity of the HIFEM/RF energy delivered to the patient to maximize the amount of energy delivered up to the point of patient comfort because more energy will make for more effective treatments. The physician or medical practitioner should ask the patient to indicate if each power level is comfortable or uncomfortable, gradually increasing or decreasing the intensity of the delivered HIFEM and RF energy according to the patient's response. The HIFEM energy causes muscle contractions and the physician or medical practitioner should ask if the contractions are comfortable or uncomfortable. The RF energy is warm when applied, and the physician or medical practitioner should ask if the temperature felt by the patient is comfortable or uncomfortable. Energy should be increased up to the point of comfort for HIFEM muscle contractions and RF temperature tolerance, thereby achieving the highest possible energy for each therapy session.

As the patient's muscles become stronger and the oropharyngeal tissue adapts to the treatment, the physician or medical practitioner can gradually increase the intensity of the HIFEM and RF therapy. This progressive approach allows the muscles to adapt to the increased stimuli without causing undue strain or discomfort. The customization of the method according to embodiments ensures that each patient receives the optimal treatment for their specific needs, providing a more effective, comfortable, and convenient solution for sleep apnea. In summary, the HIFEM therapy delivered by the device provides a non-invasive, targeted, and effective method for strengthening the muscles involved in breathing, thereby addressing one of the key factors contributing to sleep apnea.

Delivery of RF Therapy: The method further includes the generation of RF energy and delivery of the RF energy via the HIFEM/RF applicator 130 arranged in the chin and neck support 120 to the fat tissues located in the patient's airway. The RF therapy is another essential part of the treatment process. The RF generator 140b produces a radio frequency, which is delivered by the HIFEM/RF application directly towards the fat tissues located in the patient's airway. The RF therapy gencrates heat through the oscillation of charged particles within the tissues, leading to thermal damage and subsequent reduction of fat cells. The heat generated by the RF energy increases the temperature of the fat cells, causing them to break down and be naturally eliminated by the body over time. In the context of sleep apnea treatment, the RF therapy targets and reduces the fat tissues located in and around the patient's airway. By reducing the volume of these fat tissues, the device effectively creates more space in the airway. This increased space reduces the likelihood of the tongue and other soft tissues obstructing the airway during sleep, a common cause of sleep apnea events. The intensity of the RF energy can be adjusted using the control panel 160, allowing the therapy to be customized for each patient. For example, as the muscles become stronger and the oropharyngeal tissue adapts to the treatment, the physician gradually increases the intensity of the RF therapy. This progressive approach allows the muscles to adapt to the increased stimuli without causing undue strain or discomfort. This progressive approach allows the muscles to adapt to the increased stimuli without causing undue strain or discomfort. The customization of the method according to embodiments ensures that each patient receives the optimal treatment for their specific needs, providing a more effective, comfortable, and convenient solution for sleep apnea. In summary, the RF therapy delivered by the device provides a non-invasive, targeted, and effective method for reducing fat in the patient's airway, thereby addressing another key factor contributing to sleep apnea. By combining the RF therapy with the HIFEM therapy, the device offers a comprehensive and innovative solution for treating sleep apnea.

According to an embodiment, the HIFEM energy and the RF energy are concurrently applied to the patient's mouth and throat. The HIFEM energy is pulsed at between 1 Hz to 500 Hz with a continuous application of RF energy in the range of 1 MHz to 3 MHz. Treatments are 10 to 30 minutes long, depending on the patient's comfort level. Longer treatments may be applied, depending on the patient's comfort level.

The patient's treatment progress is meticulously tracked over multiple treatment sessions. Utilizing advanced algorithms, the device analyzes various parameters and adapts the intensity of the treatments accordingly. These adjustments are based on user comfort and treatment effectiveness, as determined by improvements in obstructive sleep apnea symptoms and increased respiratory muscle strength.

Completion of Treatment: The method for treating sleep apnea is completed when 30 minutes of treatment has lapsed, or when the physician or medical practitioner determines that the patient is no longer able to comfortably tolerate treatment. Upon completion of a treatment session, the device automatically shuts off the HIFEM and RF generators 140 and terminates delivery of the HIFEM and RF energy to the patient, ensuring the patient's safety. The patient is then carefully assisted from the chin and neck support 120 of the device. It is important to ensure that the patient is feeling well and not experiencing any discomfort or adverse effects from the treatment, before having the patient stand. The patient is observed for a short post-treatment period, for example, 15 minutes, under the physician's or medical practitioner's supervision. This observation period is crucial to monitor the patient for any immediate adverse reactions or complications that may arise from the treatment. During this time, the physician or medical practitioner can answer any of patient's questions about the treatment, discuss the patient's experience, and provide further instructions for post-treatment care.

Post-treatment care instructions are provided to the patient, including staying hydrated, maintaining a healthy diet, and engaging in regular physical activity to support the treatment's muscle strengthening and fat reduction effects. The patient is also advised to monitor their sleep and breathing patterns and report any significant changes to their physician.

If the patient feels well and shows no signs of adverse reactions, they are released to return home. The patient is advised to contact their healthcare provider if they experience discomfort or unusual symptoms following the treatment. This post-treatment observation and care are essential components of patient safety and satisfaction, contributing to the overall effectiveness of the treatment process.

The chin and neck support 120 and the HIFEM/RF applicator 130 should be cleaned and disinfected in preparation for the next patient, ensuring the highest hygiene and patient safety standards.

The patient's progress is tracked over multiple treatment sessions. The intensity of the treatments may be adjusted based on the patient's comfort and effectiveness, as determined by improvements in their sleep apnea symptoms.

Upon completion of the treatment course, the patient's condition is re-evaluated. If the treatment successfully reduces sleep apnea symptoms, follow-up treatments may be scheduled as needed to maintain the results. If the patient's symptoms have not improved significantly, alternative treatment options may be explored. In all cases, the goal is to provide a safe, comfortable, and effective treatment for sleep apnea, improving the patient's sleep quality and overall health.

The method for treating sleep apnea, according to an embodiment, includes the step of terminating the treatment, by the user or physician or medical practitioner, using a control button on the control panel, when the patient or physician or medical practitioner determines that the temperature and/or intensity of application of HIFEM or RF energy to the patient's mouth and throat is causing discomfort.

Accordingly, embodiments provide a targeted treatment medical approach for addressing sleep apnea. In particular, embodiments provide a device and a method providing a non-invasive treatment for sleep apnea, eliminating the need for surgical interventions or uncomfortable devices that must be worn during sleep. The device and method provide effective therapy for sleep disorders, such as sleep apnea, by combining HIFEM and RF therapies to effectively strengthen the muscles involved in breathing and reduce the amount of fat in the patient's airway. This dual approach addresses the root causes of sleep apnea, significantly improving symptoms.

Embodiments provided a customized treatment based on the patient's needs. The customized treatment includes adjusting the intensity of the HIFEM and RF therapies and selecting the correct size of the chin and neck support with an integrated HIFEM/RF applicator based on the patient's anatomy.

Embodiments provide a device for treating sleep apnea including a chin and neck support with an HIFEM/RF applicator that is configured to direct the HIFEM and RF energy directly to the patient's mouth and throat through the patient's jaw for optimizing application of the energy to the appropriate muscles controlling respiration and to the patient's airway.

Embodiments provide a device for treating sleep apnea including safety measures, such as a button for the patient or physician or medical practitioner to press if the temperature or intensity of the muscle contractions is too great for the patient's comfort, and a protocol for observing the patient post-treatment to ensure their safety.

Embodiments provide a method for treating sleep apnea that progressively apply treatment at very low, safe levels of energy and gradually increase those levels from treatment to treatment, thereby considering the patient's comfort and the progressive strengthening of the muscles' contractions.

The device and the method are designed for use in a clinical setting under the supervision of a trained healthcare professional to ensure the safety and effectiveness of the treatment. By addressing the root causes of sleep apnea, the device and the method can significantly improve patients' sleep quality, resulting in improvements to overall health and well-being and a reduction in the risks associated with untreated sleep apnea.

The device and the method offer cost-effective solutions for sleep apnea treatment, reducing the need for ongoing treatments or interventions, and thereby leading to significant cost savings for patients and healthcare providers.

The device is easy to use and the method is easy to perform, utilizing a user-friendly control panel and clear instructions for positioning and operating the device, thereby making it accessible for a wide range of healthcare providers and patients.

According to various embodiments, the device and the method can also be simplified and used within a patient's home. The in-home device includes user-friendly controls and safety features, enabling a patient to apply treatment in the comfort of their home. In-home treatment would include the guidance of a healthcare provider.

According to an embodiment, the device could be configured as a portable device for easy transport and use in various settings. A portable device is particularly useful for patients who travel frequently or for healthcare providers offering mobile services.

According to an embodiment, the device could be configured to treat other sleep disorders, such as snoring and other respiratory conditions, by adjusting the application of HIFEM and RF energy in response to the level of weakened muscles or excess fat in the patient's airway.

According to an embodiment, the device could be configured to use other energy sources or therapies to stimulate muscles and reduce fat, such as ultrasound or laser therapy.

According to an embodiment, the device could be configured to include integrated diagnostic tools, such as sensors that monitor muscle strength or airway size, providing real-time feedback on the effectiveness of the treatment.

According to an embodiment, the device could be configured to include various applicator sizes and shapes to accommodate patients with different anatomical features. The device could include applicators designed for different parts of the body or even custom-made applicators based on individual patient measurements.

According to an embodiment, the device could be configured to include a system that automatically adjusts the intensity of the treatment based on real-time feedback from sensors or patient input, thereby optimizing the effectiveness of the treatment and improve patient comfort.

According to an embodiment, the device could be operated wirelessly, eliminating the need for a power cord and increasing the ease of use and portability.

According to an embodiment, the device could be integrated with virtual or augmented reality technology to provide visual feedback or guidance during treatment, enhancing the patient experience and potentially improving treatment outcomes.

According to an embodiment, the device could incorporate artificial intelligence algorithms to optimize treatment parameters based on individual patient data, potentially improving the effectiveness and efficiency of the treatment.

According to an embodiment, the stem portion and the seat portion of the chin and neck support could be separate components that can be mixed and matched according to the patient's size and needs. This would allow for a greater range of customization and could improve the comfort and effectiveness of the treatment.

According to an embodiment, the stem portion and the seat portion of the chin and neck support could be separate components, allowing for the individual use of either the neck or chin rest. This could benefit patients who only need treatment in one of these areas or who find it more comfortable to use one piece at a time.

According to an embodiment, the device could be configured to use only RF energy to reduce fat in the airway, thereby simplifying the device, reducing cost of the device, and addressing the patient's need to reduce fat in the patient's airway, rather than muscle strengthening.

According to an embodiment, the device could be configured to use only HIFEM energy to strengthen the muscles involved in breathing, thereby simplifying the device, reducing cost of the device, and addressing the patient's need to strengthen muscles, rather than reducing fat in the patient's airway.

According to an embodiment, the device could be configured such that the HIFEM and RF generators are separate, removable modules that can be added or removed as needed. This would allow for a high degree of customization and could make the device more adaptable to the needs of different patients.

According to an embodiment, the device could include a set of interchangeable applicators of different sizes and shapes, further enabling customization of the device to accommodate different patients' anatomies and improving the comfort and effectiveness of the treatment.

According to an embodiment, the device could be used with other sleep disorder devices, such as a CPAP machine to delivery CPAP therapy, while also using this device to strengthen the airway and reduce obstructive fat deposits in and around the patient's airway. The physician or medical practitioner is also able to monitor patient pressure and AHIs on most modern CPAPs and can adjust the pressure of the CPAP down as the patient shows improved night-time respiration, or the physician or medical practitioner may leave the pressure constant and lower AHIs well below baseline in order to improve sleep quality.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that any claims presented define the scope of the various embodiments and that methods and structures within the scope of these claims and their equivalents be covered thereby.

Embodiments described herein, therefore, are well adapted to carry out the objects and attain the ends and advantages mentioned, as well as others inherent therein. While a presently preferred embodiment has been given for purposes of disclosure, numerous changes exist in the details of procedures for accomplishing the desired results. These and other similar modifications will readily suggest themselves to those skilled in the art, and are intended to be encompassed within the spirit of the various embodiments disclosed herein and the scope of the appended claims.

Although the various embodiments have been described in detail, it should be understood that various changes, substitutions, and alterations can be made hereupon without departing from the principle and scope of the various embodiments. Accordingly, the scope of the various embodiments should be determined by the following claims and their appropriate legal equivalents.

The singular forms “a,” “an,” and “the” include plural referents, unless the context clearly dictates otherwise.

Optional or optionally means that the subsequently described event or circumstances can or may not occur. The description includes instances where the event or circumstance occurs and instances where it does not occur.

Ranges can be expressed herein as from about one particular value, and/or to about another particular value. When such a range is expressed, it is to be understood that another embodiment is from the one particular value and/or to the other particular value, along with all combinations within said range.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that the mere use of the term “first” and “second” does not require that there be any “third” component, although that possibility is contemplated under the scope of the various embodiments.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The embodiments can suitably comprise, consist or consist essentially of the elements disclosed and can be practiced in the absence of an element not disclosed.

What is claimed is:

1. A device for treating sleep apnea, comprising:
a stand;
a chin and neck support;
an applicator integrated into the chin and neck support configured to provide High-Intensity Focused Electromagnetic Technology (HIFEM) therapy and Radio Frequency (RF) therapy; and
a control panel configured to adjust an intensity of the delivered HIFEM and RF therapies in real-time,
wherein the applicator is configured to provide HIFEM therapy by delivering HIFEM energy to a patient's mouth, throat, and neck to stimulate and strengthen muscles associated with the patient's breathing, and
wherein the applicator is further configured to provide RF therapy by delivering RF energy to reduce fat tissue in the patient's airway.

2. The device of claim 1, wherein the chin and neck support comprises a stem portion and a seat portion, the stem portion of the chin and neck support configured to support the patient's neck and the seat portion of the chin and neck support configured to support the patient's jaw during treatment.

3. The device of claim 1, further comprising:
one or more HIFEM and RF generators, each HIFEM generator configured to generate HIFEM energy for providing the HIFEM therapy to the patient's mouth, throat and neck, and each RF generator configured to generate RF energy for providing RF therapy to the patient's airway.

4. The device of claim 1, wherein the applicator is configured to deliver the HIFEM and RF energy concurrently to the muscles and fat tissues in the airway of the user of the device.

5. The device of claim 1, wherein the chin and neck support is selected based on the length and width of the user's neck and jaw to enhance the user's comfort and the effectiveness of treatment.

6. The device of claim 4, wherein applicator is configured to concurrently deliver the HIFEM energy pulsed at between 1 Hz to 500 Hz with a continuous application of RF energy in the range of 1 MHz to 3 MHz.

7. The device of claim 1, wherein the chin and neck support and the applicator are each curved to maintain a uniform distance to the neck throughout an HIFEM and RF energy application zone.

8. The device of claim 1, wherein the control panel comprises a patient-operated button configured to terminate the delivery of the HIFEM and RF energy, when the patient experiences discomfort during treatment.

9. The device of claim 1, wherein the applicator is configured to deliver the HIFEM energy and RF energy for a duration of 10 to 30 minutes long, depending on the patient's comfort level.

10. A method for treating sleep apnea, the method using a device, comprising:
a stand, a chin and neck support, an applicator integrated into the chin and neck support configured to provide High-Intensity Focused Electromagnetic Technology (HIFEM) therapy and Radio Frequency (RF) therapy, and a control panel configured to adjust an intensity of the delivered HIFEM and RF therapies in real-time, wherein the applicator is configured to provide HIFEM therapy by delivering HIFEM energy to a patient's mouth, throat, and neck to stimulate and strengthen muscles associated with the patient's breathing, and further configured to provide RF therapy by delivering RF energy to reduce fat tissue in the patient's airway,
the method comprising the steps of:
selecting the chin and neck support based on the length and width of the user's neck and jaw to enhance the user's comfort and the effectiveness of treatment;
generating the HIFEM and RF energy using a set of HIFEM and RF generators;
delivering the HIFEM energy from the applicator to the patient's mouth, throat, and neck through the patient's jaw to stimulate and strengthen muscles associated with the patient's breathing;
concurrently delivering the RF energy from the applicator to the patient's neck to reduce fat tissue in the patient's airway; and
adjusting the intensity of the delivering of the HIFEM energy and the RF energy based on the patient's comfort, using the control panel to treat the patient's sleep apnea.

11. The method of claim 10, wherein the delivering of the HIFEM energy is pulsed at between 1 Hz to 500 Hz with the delivering of a continuous application of RF energy in the range of 1 MHz to 3 MHz for 30 minutes.

12. The method of claim 10, further comprising:
terminating the delivery of the HIFEM energy and the RF energy, using a patient-operated button on the control panel, when the patient experiences discomfort during treatment.

13. The method of claim 10, further comprising:
treating the patient's sleep apnea using a Continuous Positive Airway Pressure device.
